(19) United States
(12) Patent Application Publication
(10) Pub. No.: US 2012/0329138 A1
(43) Pub. Date: Dec. 27, 2012

(54) PROCESS FOR SEPARATION OF A MIXTURE CONTAINING A MICROBIAL SUBSTANCE AND A LIQUID

(75) Inventors: Henry Van Kaathoven, Amsterdam (NL); Johannes Pieter Haan, Amsterdam (NL); Wilhelmus Maria Bond, Amsterdam (NL); Johannes Leendert Willem Cornelis Den Boestert, Amsterdam (NL)

(73) Assignee: SHELL OIL COMPANY, Houston, TX (US)

(21) Appl. No.: 13/330,000

(22) Filed: Dec. 19, 2011

(30) Foreign Application Priority Data
Dec. 23, 2010 (EP) 10196933.5

(51) Int. Cl. C12N 1/12; C11B 3/16; C12N 1/14; C12N 1/20; C12N 1/16

(52) U.S. Cl. 435/252.5; 435/252.1; 435/255.1; 435/252.8; 435/253.3; 435/253.1; 435/254.1; 435/256.2; 435/256.6; 435/256.1; 435/256.7; 435/256.3; 435/255.5; 435/255.6; 435/255.2; 435/255.4; 435/255.3; 435/257.1; 435/257.3; 554/175

(57) ABSTRACT

A process for separation of a mixture containing a microbial substance and a liquid using deformable filter is provided.

This patent application claims the benefit of EP10196933.5 filed 23 Dec. 2010, which is incorporated herein by reference.

### TECHNICAL FIELD OF THE INVENTION

The present invention relates to a process for separation of a mixture containing a microbial substance and a liquid.

### BACKGROUND OF THE INVENTION

With the diminishing supply of crude mineral oil, use of renewable energy sources is becoming increasingly important for the production of chemicals and fuel products. These fuel and chemical products from renewable energy sources are often referred to as biofuels, respectively biochemicals. One of the advantages of using renewable energy sources is that the CO₂ balance is more favorable as compared with a conventional energy source.

Biofuels and/or biochemicals derived from non-edible renewable energy sources are preferred as these do not compete with food production.

Oil can for example be obtained from microorganisms by secretion of the oil by the microorganisms or by extraction of the oil after cell wall disruption of the cells of the microorganisms.

Ethanol for use in a fuel composition can for example be obtained from ethanol-producing yeasts.

US2010/0184197 describes methods for harvesting biological materials, including methods for harvesting microalgal cells for biodiesel production and food or nutritional supplements, using membrane filters. The membrane filters are described to be porous monolithic bodies or supports optionally coated with at least one membrane layer. Examples of the monolithic supports include ceramic materials and carbon-based materials. These type of materials are not flexible (i.e. they are not deformable). The biological material is passed through the membrane filter for example with help of a vacuum pump or other similar mechanisms as a driving force, to force the biological material through the membrane filter. For example in the process of FIG. 2 of US2010/0184197 the biological suspension is forced through the membrane filter by a peristaltic pump on one side and a vacuum pump on the other side of the filter. Both the vacuum pump and the peristaltic pump require energy to operate. A disadvantage of the methods of US2010/0184197 is therefore the high energy consumption needed to achieve filtration. In order to break down the biological material cake that may form along the walls of the membrane, circulation may run at a higher flow rate prior to recovery, or the pump may run in reverse to collect concentrated biological material. A disadvantage of this method is that it cannot be done continuously and that it again requires extra amounts of energy.

US2007/0056902 describes a process for the treatment of septage, the septage can for example be filtered by means of a filter press. Such a filter press has chamber plates that are rigid in nature and not flexible (i.e. they are not deformable). US2004/0067574 describes a process for obtaining an oil from microbial cells. It describes that after fermentation, if necessary, liquid (usually water) can first be removed from the fermentation broth before the fermentation broth is passed to cell-wall disrupting equipment (e.g. a homogenizer). Such dewatering may be carried out by centrifugation and/or filtration. Hereafter the cell walls of the microbial cells can be disrupted to retrieve the oil.

U.S. Pat. No. 7,351,558 also describes a process for obtaining lipids from micro-organisms. It describes that typical microbial lipid manufacturing processes involve growing microorganisms in a fermentor, isolating the microorganisms, and extracting the intracellular lipids with organic solvent, e.g., hexane. U.S. Pat. No. 7,351,558 mentions that the isolation of microorganisms involves diluting the fermentation broth with water and centrifuging the mixture to isolate microorganisms.

In these processes according cultured and/or fermented microorganisms are separated from the liquid surrounding them by centrifugation, high-force filtration or drying.

Separation of the microorganisms from surrounding liquid by means of centrifugation has the disadvantages that energy consumption in the separation process is high and that the rotating parts of the centrifuge are prone to wear and require regular maintenance service. Such rotating parts may suffer considerably from foreign matter (such as for example sand) that may be present in the microbial residue. In addition extensive centrifugation may damage the cell walls of the microorganisms even when this is not desired.

Separation of the microorganisms from surrounding liquid by means of traditional filtration has the disadvantage that the filter gets clogged and needs regular cleaning service. A filter may for example get clogged by a residue that may be sticky and may contain mud (sand). Alternatively

Separation of the microorganisms from surrounding liquid by means of drying exposes the microorganisms to heat, which can damage, i.e., degrade the quality of the lipid and subsequent oil if done incorrectly, as explained in more detail in U.S. Pat. No. 7,351,558.

### SUMMARY OF THE INVENTION

It would be advantageous to provide a process for separation of a microorganism, for example an oil or ethanol producing microorganism, from the liquid surrounding them, which would require less energy and/or service and/or which is less damaging to the microorganisms.

Accordingly, in an embodiment, a process for separating a mixture containing a microbial substance and a liquid is provided, wherein the microbial substance comprises at least one microorganism that store and/or secrete ethanol or the microbial substance comprises at least one whole oleaginous microorganism and/or is obtained after lysis of at least one oleaginous microorganism, comprising:

a) bringing the mixture in contact with a deformable filter;
b) filtering the mixture with such deformable filter to form a filtrate depleted in microbial substance and a filter residue enriched in microbial substance;
c) breaking the filter residue by deformation of the filter while the mixture is in contact with said filter;
d) separating the filtrate depleted in microbial substance and the filter residue enriched in microbial substance.

### BRIEF DESCRIPTION OF THE DRAWINGS

These drawings illustrate certain aspects of some of the embodiments of the invention, and should not be used to limit or define the invention.

FIG. 1 illustrating one embodiment of a process according to the invention.

FIG. 2 illustrating another embodiment of a process according to the invention

FIG. 3 illustrating an embodiment of how the filtrate obtained by the process according to the invention can be further used.

### DETAILED DESCRIPTION OF THE INVENTION

It has now been found that a filtering process such as for example described in EP1426089 can advantageously be used for the separation of a mixture containing a microbial substance and a liquid to form a filtrate depleted in microbial substance and a filter residue enriched in microbial substance. The microbial substance can for example comprise one or more microorganisms that store and/or secrete ethanol or one or more whole oleaginous microorganisms and/or can be obtained after lysis of one or more oleaginous microorganisms,

It was furthermore found that although the mixture containing microbial substance and a liquid as described herein has a different texture and is more slippery than liquid manure originating from pigs, processing with help of a filtering process as claimed herein is still possible with good yields.

In addition to the above, it was found that the filtering process not only allows the separation of a microbial substance and water, but also advantageously allows one to separate microbial oil or ethanol from microbial substance as described herein.

EP0714318 describes a filter for separation of solids and liquids from muds and specifically muds from industrial processing. In order to separate the solids and liquids in the mud, a container is used comprising a filtering bag and a deformable membrane, comparable to fire-hoses, housed in the container. In the container, squeezing of the filtering bag is achieved by enlarging the size of the volume defined by the deformable membrane (i.e. the fire-hose). The filtering bag is provided with elastic means for ensuring elastic expansion when the squeezing phase is completed.

EP1426089 describes a method for filtering biological mud, in particular liquid manure originating from pigs. The method comprises bringing the mud in co-operative connection with a filter, generating a pressure difference over the filter causing a flow of the fluid medium through the filter and a formation of a residue comprising the solid particles on the side of the filter facing the mud, and breaking the residue while the mud is in co-operative connection with the said filter. The mud is flocculated before the pressure difference is generated, and as a filter a filter having an abhesive surface, with the abhesive surface facing the mud, is applied.

In one embodiment, the present invention provides a process for separation of a mixture containing a microbial substance and a liquid,

wherein the microbial substance comprises one or more microorganisms that store and/or secrete ethanol or the microbial substance comprises one or more whole oleaginous microorganisms and/or is obtained after lysis of one or more oleaginous microorganisms, which process comprises
a) bringing the mixture in contact with a deformable filter;
b) filtering the mixture by means of such deformable filter to form a filtrate depleted in microbial substance and a filter residue enriched in microbial substance;
c) breaking the filter residue by deformation of the filter while the mixture is in contact with said filter;
d) separating the filtrate depleted in microbial substance and the filter residue enriched in microbial substance.

The process according to the invention has a reduced energy consumption compared to the processes according to the prior art and requires less service.

In addition the use of the process according to the invention for the separation of a microbial substance and liquid allows for a continuous manner of operation. It is especially advantageous that the process according to the invention allows the filter to be continuously unplugged by continuous deformation of the filter.

Further the process of the invention is especially advantageous when used to separate microorganisms and a liquid where it is desired to avoid damage to the cell walls of the microorganisms, as damage to the cell walls of the microorganisms can be kept to a minimum.

The microbial substance or liquid separated in the process according to the invention may be retrieved and subsequently used to produce chemicals and/or fuels.

The process according to the invention separates a mixture containing a microbial substance and a liquid. The mixture can be any mixture known in the art to contain a microbial substance and a liquid. Preferably the mixture consists essentially of a microbial substance and a liquid, and optionally sand, more preferably the mixture consists essentially of a microbial substance and a liquid.

Preferably the mixture contains microbial substance in an amount in the range from 0.01 wt % to 20 wt %, more preferably in the range from 0.1 wt % to 10 wt %, most preferably in the range from 0.5 wt % to 5 wt %, based on the total weight of the mixture, the remainder being liquid and optionally sand.

By a microbial substance is herein understood a composition of matter from microbial origin. By microbial origin is herein understood comprising or made from one or more microorganisms. Preferably the microbial substance comprises one or more whole microorganisms; a microbial residue obtained after lysis of one or more microorganisms; and/or a mixture thereof. More preferably the microbial substance in the mixture comprises equal to or more than 50 wt %, still more preferably equal to or more than 70 wt % and most preferably equal to or more than 90 wt % of microbial substance chosen from the group consisting of one or more whole microorganisms; a microbial residue obtained after lysis of one or more microorganisms; and/or a mixture thereof. Most preferably essentially all microbial substance present in the mixture is microbial substance chosen from the group consisting of one or more whole microorganisms; a microbial residue obtained after lysis of one or more microorganisms; and/or a mixture thereof.

Preferably the mixture is a fermentation broth, a lysate mixture or a combination thereof.

In one preferred embodiment the mixture containing a microbial substance and a liquid is a fermentation broth produced by fermentation of one or more whole microorganisms. By fermentation is herein understood growing of micro-organisms under controlled circumstances. The microbial substance in the fermentation broth preferably comprises equal to or more than 50 wt %, more preferably equal to or more than 70 wt %, of one or more whole microorganisms, based on the total weight of microbial substance. The fermentation broth may in addition comprise less than 50 wt %, more preferably less than 30 wt %, of microbial residue, comprising disrupted cell walls of the cells of the one or more microorganisms, based on the total weight of microbial substance. For practical purposes the above percentages may be calculated on the total weight of dry matter left after drying the fermentation broth. In a most preferred embodiment the fermentation broth includes essentially only whole microorganisms and essentially no microbial residue.

In another preferred embodiment the mixture containing a microbial substance and a liquid, is a lysate mixture produced by lysis of one or more microorganisms. By lysis is herein understood disruption of the cell walls of one or more cells of the one or more microorganisms. Lysis may be achieved by any manner known by the person skilled in the art to be suitable for this purpose. For example lysis may be achieved with the help of enzymatic, physical, chemical, osmotic or mechanical methods or a mixture thereof.

In a preferred embodiment the lysate mixture contains a microbial oil, water, and a microbial residue comprising disrupted cell walls of the cells of the one or more microorganisms.

The microorganism is preferably an organism having a diameter smaller than 1 mm, more preferably a diameter smaller than 0.6 mm and still more preferably a diameter smaller than 0.4 mm. The diameter is measured at its largest point. Most preferably the microorganism comprises a diameter in the range from 0.5 to 200 micrometer, even more preferably in the range from 1 to 100 micrometer.

A wide variety of microorganisms can be used in the microbial substance. The one or more microorganisms can for example include autotrophic and/or heterotrophic microorganisms. By an autotrophic microorganism is herein understood a microorganisms that obtains carbon by fixing carbon dioxide. By an heterotrophic microorganism is herein understood a microorganism that obtains carbon from organic carbon sources, such as for example carbohydrates. The energy metabolism of the microorganism can for example be based on phototrophy (that is where the microorganism uses light as its source of energy) or it can be based on chemotrophy (that is where the microorganism uses organic substances as its source of energy). The one or more microorganisms can include aerobic and/or anaerobic microorganisms.

The microorganisms are preferably unicellular. The microorganisms may be present as separated unicellular microorganisms or as a colony of unicellular microorganisms. The one or more microorganisms can include so-called wild-type microorganisms and/or genetically modified microorganisms.

The one or more microorganisms can include microorganisms that merely store microbial oil and/or microorganisms that secrete microbial oil.

Micro-organisms that store and/or secrete microbial oil are sometimes also referred to as oleaginous micro-organisms. The microbial substance preferably comprises, and more preferably consists of, one or more whole oleaginous microorganisms and/or the microbial residue obtained after lysis of one or more oleaginous microorganisms. Such an oleaginous microorganism preferably is able to store oil in its interior in an amount of equal to or more than 10 wt %, more preferably equal to or more than 15 wt %, still more preferably equal to or more than 20 wt % and most preferably equal to or more than 30 wt % of its dry biomass. In a preferred embodiment the microbial substance is a composition of matter from an oleaginous microbial origin. By an oleaginous microbial origin is herein understood comprising or made from one or more oleaginous microorganisms.

Hence in a preferred embodiment essentially all microbial substance present in the mixture is microbial substance chosen from the group consisting of one or more whole oleaginous microorganisms; a microbial residue obtained after lysis of one or more oleaginous microorganisms; and/or a mixture thereof. Alternatively the one or more microorganisms can include microorganisms that store and/or secrete ethanol. Examples include ethanol-secreting yeasts and bacteria.

Preferably the microbial substance comprises one or more microorganisms chosen from the group consisting of bacteria, fungi, yeasts, algae and mixtures thereof.

Examples of bacteria include Rhodococcus, Mycobacteria, Vibrio, Escherichia coli, Bacillus, Brevibacterium, Corynebacterium, Flavobacterium, Klebsiella, Micrococcus, Mycoplana, Paracoccus, Pseudomonas, Acetobacter and/or other bacteria and/or genetically modified strains thereof and/or mixtures thereof.

In one preferred embodiment the one or more microorganisms include fungi. Examples of such fungi include fungi of the genera Saprolegnia, Phytophthora, Mucor, Rhizopus, Absidia, Mortierella, Cunninghamella, Taphrina, Monascus, Nectria, Gibberella, Chaetomium, Neurospora, Geotrichum, Monilia, Trichoderma, Aspergillus, Penicillium, Paecilomyces, Gliocladium, Sporotrichum, Microsporum, Trichophyton, Cladosporium, Syncephalastrum, Phycomyces and Eupenicillium and/or genetically modified strains thereof and/or mixtures thereof.

In another preferred embodiment the one or more microorganisms include yeasts. Yeasts are eukaryotic micro-organisms classified in the kingdom Fungi. A wide range of yeasts can be used, including for example yeasts of the genera Endomyces, Shizosaccharomyces, Pichia, Hansenula, Debaryomyces, Saccharomyces, Saccharomycopsis, Rhodotorula, Sporobolomyces, Cryptococcus, Candida, and Brettanomyces, Lipomyces, Endomycopsis, Rhodosporidium, Yarrowia and/or genetically modified strains thereof and/or mixtures thereof.

For example an ethanol storing and/or secreting microorganisms may be chosen from the group consisting of Saccharomyces spp., Saccharomyces cerevisiae, Escherichia, Zymomonas, Candida, Pichia, Streptomyces, Bacillus, Lactobacillus, Clostridium, Myceliophthora and mixtures thereof.

In another preferred embodiment the one or more microorganisms include algae, most preferably microalgae. By microalgae are herein understood unicellular microorganisms capable of photosynthesis. Such microalgae include for example green algae and cyanobacteria, but exclude seaweed. More preferably the microorganisms are eukaryotic microalgae.

The algae may be sweet water algae or salt water algae. Preferably, the microalgae used in the process of the present invention are marine microalgae.

Suitable marine microalgae can include members from various divisions of algae, including for example diatoms, pyrrophyta, nitszchia, porphyridia, ochrophyta, chlorophyta, euglenophyta, dinoflagellata, chrysophyta, crypthecodinia, phaeophyta, rhodophyta and cyanobacteria. Preferably, the marine microalgae are members from the diatoms or ochrophyta division, more preferably from the raphid, araphid, and centric diatom family.

Examples of suitable algae include Botryococcus braunii, Chlorella, Dunaliella tertiolecta, Gracilaria, Pleurochrysis carterae and/or genetically modified strains thereof and/or mixtures thereof.

The one or more microorganisms can be produced by cultivation and/or fermentation in any manner known by the skilled in the art to be suitable for this purpose. For example the cultivation and/or fermentation of the one or more microorganisms can be carried out under open culture conditions or closed culture conditions.

If the microbial substance contains oleaginous yeasts, such oleaginous yeasts are preferably fermented under closed-culture conditions, preferably in closed bioreactors. The yeasts can be fermented at a wide range of fermentation conditions. Preferably the yeasts are fermented at a pH condition in the range from 2.5 to 7, more preferably in the range from 3 to 5. The temperature during fermentation preferably lies in the range from 20° C. to 40° C., more preferably in the range from 25° C. to 35° C. During fermentation, the bioreactor is preferably aerated to a saturation level in the range of 1 to 50%, more preferably 10 to 30% of the complete saturation of the water with air. The oleaginous yeasts can be fermented in any culture medium known by those skilled in the art to be suitable for this purpose. Preferably the culture medium comprises a carbon source. A preferred carbon source are sugars. These sugars can include monosaccharides, disaccharides, trisaccharides and polysaccharides. Examples of sugars that can be comprised in the culture medium include for example fructose, galactose, glucose, sucrose, xylose, maltose, sacharose, lactose, dextrose and corn syrup. In an especially preferred embodiment sugar cane, more preferably Brazilian sugar cane is used as a carbon source. The carbon source is preferably present in a concentration of from 2 to 35 wt %, more preferably in a concentration of from 5 to 25 wt %. The oleaginous yeasts are preferably present in a concentration of 1 to 20 wt %, more preferably in a concentration of 5 to 15 wt %.

The microorganisms can be grown in various manners, including in a continuous mode, in a continuous mode with a recycle of microorganism cells, batch-wise, or in a fed-batch mode (wherein growth of the microorganism is controlled by feeding a controlled amount of nutrient substrate to the culture medium).

The microorganisms can be cultivated under open or closed culture conditions. Closed culture conditions may include for example closed photo-bioreactors (that is a closed bioreactor that allows for example microalgae to have access to light). Open-culture conditions may include for example open sea or ponds. For example microalgae can be cultivated in freshwater, sweet water, saline water or moist earth.

The mixture containing a microbial substance and a liquid may contain any liquid known by the skilled person to exist in a fermentation broth or other mixture of microbial substance and liquid. The mixture may contain for example microbial oil, microbial ethanol and/or water.

In a preferred embodiment the liquid contains water. More preferably the liquid contains equal to or more than 30 wt % of water, still more preferably equal to or more than 50 wt % of water and most preferably equal to or more than 70 wt % of water. Although there is no upper limit, for practical purposes the liquid may preferably contain equal to or less than 100 wt % water, more preferably equal to or less than 99 wt % water and most preferably equal to or less than 95 wt % water.

In another preferred embodiment the liquid contains water and/or ethanol.

In still another preferred embodiment the liquid contains water and/or microbial oil.

By a microbial oil is herein understood an oil from microbial origin. The microbial oil may for example be obtained via secretion by one or more oil-secreting microorganisms and/or via extraction from one or more oil-storing microorganisms.

The microbial oil preferably contains one or more lipids. Preferably the lipids comprise naturally occurring compounds that are essentially hydrophobic in nature and contain long-chain aliphatic hydrocarbons. The one or more lipids preferably comprise monoglycerides, diglycerides and/or triglycerides (which are mono- di- and tri-esters of glycerol and fatty acids); phospholipids (which are esters of glycerol and phosphate group-substituted fatty acids); and glycolipids (which are esters of fatty acids and sugars).

The fatty acid moiety in the one or more lipids can comprise saturated fatty acids and/or unsaturated fatty acids containing one, two, three or more double bonds.

The microbial oil can further contain a wide range of additional compounds. For example, the microbial oil can further contain (saturated or unsaturated) free fatty acids and/or esters thereof; fatty alcohols and/or fatty amines; carotenoids; terpenes; styrols; tocopherols; and/or proteins.

If the liquid contains a microbial oil, the liquid preferably contains equal to or more than 0.5 wt % of microbial oil, still more preferably equal to or more than 10 wt % of microbial oil and most preferably equal to or more than 50 wt % of microbial oil. For practical purposes the liquid may preferably contain equal to or less than 99 wt % of microbial oil, more preferably equal to or less than 70 wt % of microbial oil and most preferably equal to or less than 50 wt % of microbial oil.

If the liquid contains ethanol, the liquid preferably contains equal to or more than 0.5 wt % of ethanol, still more preferably equal to or more than 10 wt % of ethanol and most preferably equal to or more than 50 wt % of ethanol. For practical purposes the liquid may preferably contain equal to or less than 99 wt % of ethanol, more preferably equal to or less than 70 wt % of ethanol and most preferably equal to or less than 50 wt % of ethanol.

In addition to the microbial substance and the liquid, the mixture to be separated in the process of the invention may optionally contain one or more other components. Examples include sand and several gasses.

In one preferred embodiment the mixture to be separated in the process according to the invention consists essentially of microbial substance and water.

In another preferred embodiment the mixture to be separated in the process according to the invention consists essentially of microbial oil, microbial substance and water. For example the mixture to be separated may consist of an aqueous suspension of microbial oil and microbial substance.

In a further preferred embodiment the mixture to be separated in the process according to the invention consists essentially of microbial substance, ethanol and water.

In a preferred embodiment, the mixture containing microbial substance and liquid is subjected to a concentration step before use in step a) of the process according to the invention. Such a concentration step conveniently provides a concentrated mixture containing microbial substance and liquid. The concentration step has the advantage that the volume of material to be processed in step a) is reduced and less energy is needed to process large amounts of solvent (for example water) in or after step a). The mixture may for example be concentrated with help of coagulation, flocculation and/or flotation or sedimentation, and/or by making use of a cyclone-type, PARC-type or Lakos-type separation unit.

Preferably the mixture containing microbial substance and liquid is at least concentrated by means of coagulation and/or flocculation before being contacted with the filter in step a).

Coagulation can be achieved by adding a coagulant to the mixture of microbial substance and liquid. By a coagulant is understood a substance or mixture of substances that is capable of coagulating (sticking together) in particular colloidal particles, i.e. particles that have a maximum diameter of approximately 1 mm. Because of their small size, these particles tend to run through the filter, together with the liquid and contaminate the filtrate. The coagulant can advantageously be added to make these small particles aggregate to bigger ones which are capable of being flocculated into flakes as described below. In this way a very clean filtrate can be obtained.

Any coagulation step is preferably carried out by rapid mixing of the coagulant and the mixture of microbial substance and liquid during at least 1 minute and preferably during at least 3 minutes. Any coagulant known by the skilled person to be suitable for coagulation of microbial substance can be used. Preferred coagulants include aluminium and iron salts. Examples of suitable coagulants include, hydrated potassium aluminum sulfate, aluminium chlorohydrate, aluminium sulfate, ferric chloride, ferrous sulfate, ferric sulfate and/or sodium aluminate. Chloride salts of aluminium and iron are most preferred.

By flocculation is understood treating the mixture such that flakes of aggregated solid particles in the mixture are provided. Flocculation can be achieved by adding a flocculant to the mixture of microbial substance and liquid. By a flocculant is understood a substance or mixture of substances, which is capable of providing flakes of aggregated solid particles. Any flocculant known by the skilled person to be suitable for flocculating microbial substance can be used. Suitable flocculants include aluminium and iron salts, such as hydrated potassium aluminum sulfate, aluminium chlorohydrate, aluminium sulfate, ferric chloride, ferrous sulfate, ferric sulfate and/or sodium aluminate. Other suitable flocculants include both anionic (negatively charged) polymers and cationic (positively charged) polymers. Anionic polymers are preferably used in combination with metal containing coagulants. Cationic polymers may be used alone or in combination with the aluminum and iron type coagulants as listed above.

Preferred flocculants are high molecular weight cationic polymers, more preferably cationic polymers having a molecular weight of equal to or more than 10.000 Dalton. Such flocculants may be used alone or in combination with a coagulant. Most preferred flocculants include SYNTHOFLOQ 5010HL available from Breustedt Chemie, or Nalco 71303, Nalco ShellCore 71301, Nalco ShellCore 71325, Nalcolyte 8100, Nalco C-6287.

If it is intended to use the filter residue for animal feed applications, it is further preferred to use a coagulant and/or flocculant that is GRAS (Generally Regarded As Save)-certified.

The coagulants and/or flocculants are preferably mixed with the mixture containing a microbial substance and a liquid in a mixing unit. This mixing unit may for example comprise a stirred mixer or static mixer. Any coagulants and/or flocculants are preferably added to the mixture containing microbial substance and liquid in a concentration of from equal to or more than 0.1 ppm weight to equal to or less than 1 wt %, more preferably from equal to or more than 1 ppm weight to equal to or less than 0.5 wt %. The flocculation step preferably takes in the range from equal to more than 10 minutes, more preferably equal to more than 15 minutes, to equal or less than 2 hours, more preferably equal to less than 1 hour.

If a coagulant is used, such coagulant is preferably used in a weight ratio of coagulant to flocculant in the range from 1000 to 1 (1000:1) to 1 to 1000 (1:1000).

The flakes obtained by the flocculation should preferably last at least for the duration of the filtering step. Preferably the flakes obtained by the flocculation have a particle size with a diameter (measured at its largest point) in the range of equal to or more than 10 micrometer, more preferably equal to or more than 0.1 millimeter to equal to or less than 15 millimeter, more preferably equal to or less than 10 millimeter, most preferably equal to or less than 5 millimeter.

Preferably the mixture containing microbial substance and liquid is at least concentrated by sedimentation or flotation. Most preferably a concentration of the mixture containing microbial substance and liquid comprises coagulation and/or flocculation as described above, followed by a subsequent sedimentation or flotation to separate a concentrated mixture of microbial substance and liquid from the remainder of the liquid. A preferred flotation method is dissolved air flotation (DAF), where the microbial substance in the mixture is flotated to a top layer with the help of air. This top layer can subsequently be skimmed to obtain a more concentrated mixture.

When the mixture is concentrated by means of sedimentation, a bottom layer is formed which can be tapped from the bottom of a vessel. Preferably sedimentation is carried out in so-called circular thickeners, such circular thickeners suitably comprise a circular sedimentation vessel and a conically shaped bottom part from which the sediment can be retrieved.

Alternatively or in addition to the above, the mixture containing microbial substance and liquid may be concentrated by means of a PARC and/or Lakos separator.

When the one or more microorganism(s) include fungi and/or yeasts, coagulation and/or flocculation may be possible but not necessary, and a concentrated mixture advantageous for feeding to step a) can for example be obtained by mere flotation and/or sedimentation.

When the one or more microorganism(s) include algae or bacteria, it is advantageous to first subject the mixture to coagulation and/or flocculation, subsequently subject the mixture to flotation and/or sedimentation and only thereafter feed the mixture to step a).

The mixture containing microbial substance and liquid may optionally also be concentrated by means of a prefiltering device. Such a prefiltering device preferably comprises a coarse filter, for example made from a polymeric material or a metal such as stainless steel, which allows the mixture to slide from one location to another location whilst simultaneously allowing the mixture to loose already part of its liquid. Preferably the prefiltering device comprises a coarse filter from a polymeric material. The mesh size of the prefiltering device should preferably be such that at least part of the liquid in the mixture can pass the pores in the prefiltering device with the help of gravity alone. For practical purposes a mesh size in the range from equal to or more than 100 micrometer to equal to or less than 1000 micrometer, more preferably equal to or less than 500 micrometer may be preferred.

Such a prefiltering device may optionally be used before any coagulation, flocculation, flotation and/or sedimentation takes place, thereby advantageously reducing the amount of liquid that needs to be processed through such downstream units.

The prefiltering device may also optionally be used after any coagulation, flocculation, flotation and/or sedimentation and before the actual filtering according to the process of the invention, thereby advantageously allowing the concentrated mixture prepared by coagulation, flocculation, flotation and/or sedimentation to to loose already part of its liquid before filtering.

In step a) of the process according to the invention the mixture is brought in contact with a filter.

By bringing the mixture in contact with the filter is herein understood that the mixture is brought into contact with the filter in such a manner that the liquid can be filtered through said filter. Such a contact is sometimes also referred to as a co-operative connection.

The mixture that is fed into step a) preferably has a dry matter content in the range from 0.01 wt % to 10 wt %, more preferably in the range from 0.1 wt % to 8 wt %, most preferably in the range from 0.5 wt % to 4 wt %.

Preferably the filter is a deformable filter. By a deformable filter is understood a filter that can undergo deformation as described in more detail below. By deformation of the filter is herein preferably understood that the shape of the filter is changed. Preferably the filter is a filter of which the shape can be changed when, preferably mechanical, force is applied to it. Such a deformable filter is sometimes also referred to as a flexible filter.

The filter can be made from a variety of fabrics. Preferred fabrics are woven fabrics. Examples of suitable fabrics include polyamides (such as for example nylon), polyaramides (such as for example Kevlar), polypropylene, polyethylene, polyester, PET, Teflon-type polymers (such as for example PTFE and/or polytetrafluorethylene), cotton and/or mixtures thereof.

Preferably the filter is a filter having an abhesive surface, the abhesive surface facing the mixture, as described for example in EP1426089 and herein incorporated by reference. By an abhesive surface is herein understood that the surface is capable of preventing or reducing adhesion to it's surface, that is upon an impact the residue that adheres to the filter substantially comes off.

Any filter material known by the skilled person in the art to have such an abhesive surface can be used. Preferably the filter comprises an abhesive filtering fabric, such as fabrics made from materials that are known for reducing adhesion such as polymers containing a high fluorine content.

It is further preferred that the filter has a calendared surface. By calendaring the surface of the filter, sharp edges, bulges, irregularities etc. of this surface are substantially removed. The surface of the filter becomes more or less flat so that there are hardly any sites for the residue to mechanically adhere. Thus, by calendaring the surface of a filter, this surface can be made more abhesive.

In a preferred embodiment the filter comprises a first filtering fabric that is calendared and a second supporting fabric for supporting this first filtering fabric. By providing a first filtering fabric and a second supporting fabric, good filtering properties could be provided for by the first filtering fabric (preferably made out of thin fibres and having a small mesh size) and good mechanical strength could be provided for by the second supporting fabric (preferably made out of thick wires and having a large mesh size in order to be strong but not block the filter).

Preferably the filter (or if two filtering fabrics are present the first filtering fabric) comprises a mesh size ranging from equal to or more than 5 micrometer, more preferably from equal to or more than 10 micrometer, to equal to or less than 1000 micrometer, more preferably to equal to or less than 200 micrometer, and most preferably equal to or less than 100 micrometer.

For example, the mesh size may be such that a particle having a diameter of less than 5 micrometer, more preferably a particle having a diameter of less than 10 micrometer, may still pass the filter, whilst a particle having a diameter of equal to or more than 1000 micrometer, more preferably a particle having a diameter of equal to or more than 200 micrometer, most preferably a particle having a diameter of equal to or more than 100 micrometer will be retained by the filter.

The filter may have any shape known by the skilled person to be suitable for this purpose. For example the filter may be shaped as an essentially vertically arranged tube. Preferably, however, the filter is shaped as an essentially horizontally arranged belt or as an essentially horizontally arranged tube.

If the filter is shaped as an essentially horizontally arranged belt, the belt should be sufficiently broad to avoid leakage of the mixture along the sides of the belt during filtration. Preferably the belt has a width in the range from equal to or more than 1 meter, more preferably equal to or more than 2 meters, to equal to or less than 8 meters, more preferably equal to or less than 5 meters.

Alternatively, the belt may have a ridge on each side, to avoid leakage of the mixtures. Preferably such a ridge has a height in the range from equal to or more than 0.5 cm, more preferably equal to or more than 1 cm, to equal to or less than 10 cm, more preferably equal to or less than 2 cm.

In step b) of the process according to the invention, the mixture is filtered by means of such filter to form a filtrate depleted in microbial substance and a filter residue enriched in microbial substance.

Step b) of the process can be carried out in a batch, semi-batch or continuous manner. In a preferred embodiment step b) of the process is carried out in a continuous manner.

Preferably the mixture is filtered with the filtrate flowing in an essentially vertical direction. That is, preferably the filter is situated essentially horizontally, with gravitational forces assisting in the filtration and the retrieval of the filtrate.

Preferably the filter is arranged to move continuously over a conveyer belt thus allowing continuous filtration of the mixture. If the filter is arranged to move continuously over a conveyer belt, it preferably moves with a speed in the range from equal to or more than 0.001 m/s, more preferably from equal to or more than 0.01 m/s, to equal to or less than 0.5 m/s, more preferably equal to or less than 0.1 m/s.

In a preferred embodiment the mixture of microbial substance and liquid is supplied to the conveyer belt via a prefiltering device, allowing the mixture to slide onto the conveyer belt and simultaneously allowing the mixture to loose already part of its liquid as explained herein before.

Alternatively, step b) can be carried out in a continuous manner by a system of multiple horizontally and/or vertically arranged filters which are used in an alternating manner. In such a system, advantageously one or more filter(s) may be in use to filter the mixture, whereas one or more other filter(s) may be filled up, emptied or cleaned.

The above described continuous modes of operation have the advantage that the process is easy to scale up, a very important requirement for commercial use of micro-organisms in the production of biofuels and/or biochemicals.

Although a wide range of temperatures can be applied, preferably the process is carried out at ambient temperature (about 20° C.).

Preferably in step b) a pressure difference is applied over the filter. This pressure difference may assist in causing a flow of liquid through the filter and formation of the filter residue on the side of the filter facing the mixture. A wide range of pressures can be applied. Preferably a pressure difference in the range from equal to or more than 0.1 bar, more preferably equal to or more than 1 bar; to equal to or less than 15 bar, more preferably equal to or less than 10 bar, is applied. Conveniently the mixture may be pressurized with a plunger pump that is capable of pressurizing the mixture without exerting too much mechanical forces on the mixture.

During step b) the mixture is separated into a filtrate depleted in microbial substance and a filter residue enriched in microbial substance.

Preferably the filtrate contains equal to or less than 0.05 wt % of microbial substance, more preferably less than 100 ppm weight and most preferably less than 10 ppm weight microbial substance.

Preferably the filter residue contains equal to or more than 10 wt % microbial substance, more preferably equal to or more than 20 wt % microbial substance, still more preferably equal to or more than 30 wt % microbial substance and most preferably equal to or more than 40 wt % microbial substance, wherein microbial substance can be determined by determining dry matter.

In line with step c) of the process according to the invention, the filter residue is broken while the mixture is in contact with said filter. This advantageously enhances solid-liquid separation. The contact of the mixture with the filter is sometimes also referred to as a co-operative connection.

Breakage of the filter residue can be achieved by deformation, preferably mechanical deformation, of the filter. Hence, breakage of the filter residue can be achieved by changing the shape of the filter, for example by applying mechanical force to it. Preferably breakage of the filter residue is achieved by continuous deformation (i.e. continuously changing the shape) of the filter. In a preferred embodiment, filtering in step b) and breaking of the filter residue in step c) can both be carried out in a continuous manner. Such continuous breaking of the filter residue advantageously allows one to filter the mixture and unplug the filter at the same time. Hence, in a preferred embodiment steps b) and c) are carried out simultaneously.

The filter can be deformed by any means known to the skilled person for this purpose. Deformation may occur continuously or intermittently. The deformation may include peristaltic movements, kneading, vibrations, and/or combinations thereof.

Preferably the deformation includes peristaltic movements. By peristaltic movement of the filter is preferably understood an essentially symmetrical compression and relaxation of the filter. As a consequence of such essentially symmetrical compression and relaxation of the filter, the filter residue may be broken and propagated over the filter. The, preferably peristaltic, deformation may conveniently be brought about by using air filled fire hoses and/or mechanical rolls. Preferred manners of deformation are described in EP0714318 and EP1426089 and are incorporated herein by reference.

The deformation of the filter preferably has an amplitude in the range from 0.5 to 2 times the thickness of the filter residue. When the filter is an essentially horizontally arranged belt, the thickness of the filter residue may be in the range from 5 mm to 2 cm.

When the filter is an essentially vertically arranged tube, the diameter of the tube may be in the range from 10 to 50 cm.

Step c) can advantageously be operated such that essentially no lysis of the cell walls of one or more whole microorganisms takes place.

If desired, however, step c) may be designed such that one or more whole microorganisms having weak or weakened cell walls can be broken, allowing one to carry out lysis of one or more whole microorganisms in-situ during the separation process.

In step d) the filtrate depleted in microbial substance and the filter residue enriched in microbial substance are separated.

If the filtrate contains ethanol, such ethanol may advantageously be separated from any water that may also be present in the filtrate. Hence in a preferred embodiment the present invention provides a process for separation of a mixture containing a microbial substance, water and/or ethanol, which process comprises

i) bringing the mixture in contact with a deformable filter;
ii) filtering the mixture by means of such a deformable filter to form a filtrate containing water and/or ethanol and a filter residue containing microbial substance;
iii) breaking the filter residue by deformation of the filter while the mixture is in contact with said filter;
iv) separating the filtrate and the filter residue; and
v) optionally separating ethanol from the filtrate.

Preferably steps ii) and iii) are carried out continuously and/or simultaneously.

The ethanol obtained in such a process can advantageously be used as a fuel component.

If the filter residue contains one or more whole oleaginous microorganisms, the filter residue may be dried and/or subjected to a lysis step and/or subjected to an extraction step to obtain a microbial oil.

The filter residue may optionally be dried before any subsequent lysis or extraction step. Preferably drying comprises one or more solar drying steps and/or one or more forced air flow drying steps. Solar drying may conveniently comprise heating the filter residue by solar energy in a glass construction.

Preferably a filter residue containing one or more whole oleaginous microorganisms is subjected to a subsequent lysis step to obtain a lysate mixture containing microbial residue, microbial oil and optionally water. Lysis may be achieved by any manner known by the person skilled in the art to be suitable for this purpose. For example lysis may be achieved with the help of enzymatic, physical, chemical, osmotic or mechanical methods or a mixture thereof.

An example of a physical method is the heating and/or drying of the microorganisms at an elevated temperature suitable to rupture the cell walls of one or more cells of the microorganisms. In this case the microorganisms are preferably heated to a temperature of equal or more than 50° C., more preferably of equal or more than 75° C., still more preferably of equal or more than 100° C. and most preferably of equal or more than 120° C. Preferred physical methods to achieve lysis include for example boiling or steam treatment of the feed comprising one or more microorganisms.

An example of an osmotic method is the treatment of the one or more microorganisms in a hypotonic environment where the surrounding fluid has a lower salt concentration than the interior of the cells of the one or more microorganisms.

Examples of chemical methods include the treatment of the feed comprising one or more microorganisms with a base or acid or a surfactant or detergent. By a base is understood any compound whose pKa is greater than that of water. In a preferred embodiment a base is used selected from the group consisting of hydroxides, carbonates and bicarbonates of lithium, sodium, potassium, calcium and magnesium. By an acid is understood any compound whose pKa is smaller than that of water. In a preferred embodiment an acid is used selected from the group consisting of sulphuric acids, phosphoric acids, hydrochloric acid, formic acid, acetic acid, citric acid.

An example of an enzymatic method includes treatment of the feed comprising one or more microorganisms with cell wall degrading enzymes. Examples of cell wall degrading enzymes include proteases, cellulases, hemicellulases, chitinases and/or pectinases.

An example of a mechanical method is the treatment of the feed comprising one or more microorganisms with ultrasound. In such a case preferably a frequency in the range from 20-50 KHz is applied. Another example of a mechanical method is the treatment of the feed comprising one or more microorganisms with a high-shear device (also sometimes referred to as a homogenisation), such as for example a rotor-stator disruptor or valve type processor. Other examples of homogenisation include treatment with a ball-mill or bead-mill (e.g. including sand and/or glass beads).

A most preferred example of a mechanical lysis method is extrusion.

After lysis, the microbial oil can be separated from the lysate mixture by any manner known by the skilled person to be suitable for this purpose. For example, the microbial oil can be separated from the lysate mixture with the help of centrifugation, (membrane) filtration, coagulation and/or flocculation, flotation or sedimentation and/or by means of a cyclone.

The retrieved microbial oil can advantageously be used for the production of chemicals and fuel products as described in more detail below.

In one preferred embodiment the filter residue can contain microbial residue comprising disrupted cell walls of the cells of one or more microorganisms. Preferably at least part of this microbial residue is retrieved from the filter residue and used as animal feed (including fish feed) or as a feedstock to produce methane. The methane can advantageously be used as a burning fuel. In some cases it may further be advantageous to burn the retrieved filter residue or microbial residue as such. Before using any parts of the filter residue as animal feed or directly or indirectly as burning fuel, the filter residue may be washed one or more times to retrieve any residual microbial oil, with water or an extractive solvent as described herein below. Hereafter the microbial residue may preferably be dried.

In some cases the mixture containing microbial substance and a liquid used in step a) may already contain a microbial oil, for example if the mixture is a lysate mixture. In other cases the mixture in step a) is treated such during steps b) and c) that microbial oil is produced. In these cases, the filtrate may contain microbial oil.

When the filtrate contains microbial oil, the filtrate is preferably subjected to a subsequent retrieval step to retrieve the microbial oil from the filtrate.

The microbial oil may be retrieved from the filtrate in any manner known by the skilled person to be suitable for this purpose. Suitable methods for retrieval of the microbial oil from the filtrate may for example include phase separation, solvent distillation, extraction, centrifugation and separation by means of a cyclone type separation unit. When extraction is used, suitable extractive solvents include C₁-C₁₀ alkyl esters, such as ethyl acetate or butyl acetate; toluene; C₄-C₁₀ alcohols, such as for example pentanol, hexanol, ethyl hexanol; C₃-C₈ alkanes, such as for example hexane or heptanes.

In a preferred embodiment, the invention provides a process for separation of a mixture containing a microbial substance, water and a microbial oil, which process comprises

i) bringing the mixture in contact with a filter;
ii) filtering the mixture by means of such filter to form a filtrate containing water and microbial oil and a filter residue containing microbial substance;
iii) breaking the filter residue by deformation of the filter while the mixture is in contact with said filter;
iv) separating the filtrate and the filter residue.

Preferably the mixture containing a microbial substance, water and a microbial oil comprises a lysate mixture, more preferably a lysate mixture produced in situ in the process for separation by mechanical lysis of one or more whole microorganisms.

Such lysate mixture preferably contains in the range equal to or more than 0.01 wt %, more preferably equal to or more than 0.1 wt % and most preferably equal to or more than 1 wt %, to equal to or less than 100 wt %, preferably equal to or less than 80 wt %, most preferably equal to or less than 60 wt % of microbial oil, based on the total amount of liquid present in the lysate mixture.

In a preferred embodiment of the above process step ii) additionally includes washing the filter residue by adding an additional liquid during filtering. The additional added liquid can comprise water and/or one or more extractive solvents as described above.

In a further preferred embodiment, the microbial oil and the remaining liquid in filtrate are retrieved in a collector and separated by phase separation.

The retrieved microbial oil can advantageously be used for the production of chemicals and/or fuel products.

In a preferred embodiment the retrieved microbial oil is hydrotreated in a further step to produce a hydrocarbon product. Such hydrotreatment can for example include hydrogenation and/or hydrodeoxygenation and/or hydroisomerization.

In another preferred embodiment the retrieved microbial oil is enzymatically converted in a further step to produce a hydrocarbon product.

In a further preferred embodiment the hydrocarbon product produced is blended with one or more other components to produce a fuel composition.

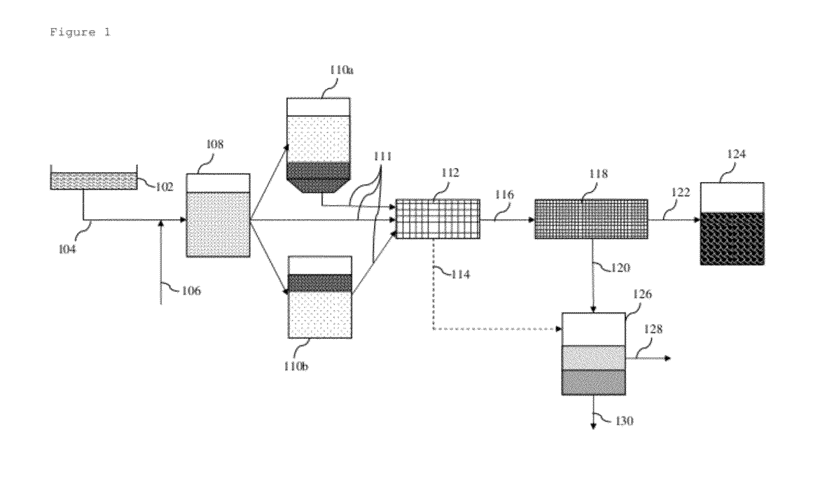

The process according to the invention can be carried out in a batchwise, semi-batchwise or continuous manner. Preferably the process according to the invention is carried out in a continuous manner. The use of the process according to the invention advantageously allows for such a continuous operation of a filter. Examples of the process according to the invention has been illustrated by non-limiting FIGS. 1, 2 and 3.

In FIG. 1 a mixture (104) comprising algae and water is obtained from an open pond (102). A combination of coagulants and/or flocculants (106) is added to the mixture (104) and the mixture is allowed to coagulate and flocculate in mixing unit (108). The contents of mixing unit (108) can be forwarded to a sedimentation unit (110a); or a flotation unit (110b); or flotation or sedimentation can be carried out in situ in mixing unit (108) after stirring has stopped. A concentrated mixture of algae and water (111) is tapped from the bottom of sedimentation unit (110a); skimmed from the top of flotation unit (110b); or tapped or skimmed from respectively the bottom or top of mixing unit (108) after stirring has stopped. The concentrated mixture of algae and water (111) is forwarded to a coarse filter (112) of stainless steel, where the mixture is allowed to loose part of the water (114). Subsequently the even more concentrated mixture (116) is forwarded to a filter (118) comprising a pore diameter of about 100 micrometer to produce a filtrate (120) and a filter residue (122). The filter residue is collected in vessel (124). The filtrate (120) and optionally the water (114) are collected in a separation vessel (126) to be phase separated in an oily phase (128) and an aqueous phase (130).

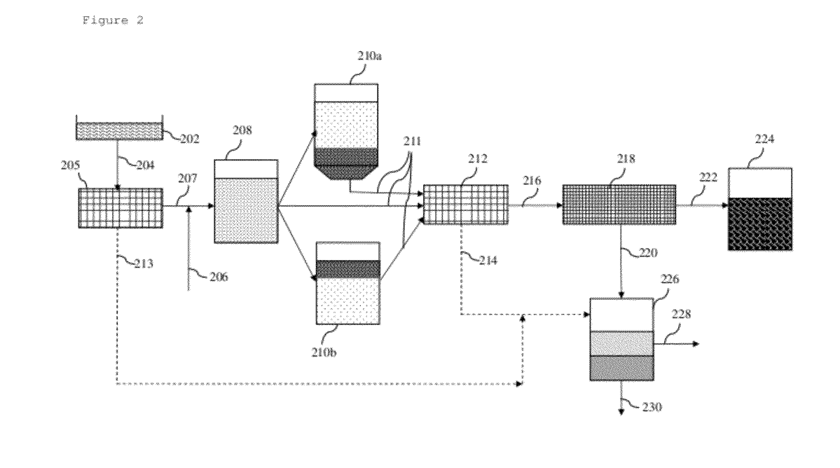

In FIG. 2 a mixture (204) comprising algae and water is obtained from an open pond (202). The mixture of algae and water (204) is forwarded to a first coarse filter (205) of stainless steel, where the mixture is allowed to loose part of the water (213). A combination of coagulants and/or flocculants (206) is added to the more concentrated mixture (207) and the mixture is allowed to coagulate and flocculate in mixing unit (208). The contents of mixing unit (208) can be forwarded to a sedimentation unit (210a); or a flotation unit (210b); or flotation or sedimentation can be carried out in situ in mixing unit (208) after stirring has stopped. A concentrated mixture of algae and water (211) is tapped from the bottom of sedimentation unit (210a); skimmed from the top of flotation unit (210b); or tapped or skimmed from respectively the bottom or top of mixing unit (208) after stirring has stopped. The concentrated mixture of algae and water (211) is forwarded to a second coarse filter (212) of stainless steel, where the mixture is allowed to loose part of the water (214). Subsequently the even more concentrated mixture (216) is forwarded to a filter (218) comprising a pore diameter of about 100 micrometer to produce a filtrate (220) and a filter residue (222). The filter residue is collected in storage vessel (224). The filtrate (220) and optionally the water (214) are collected in a separation vessel (226) to be phase separated in an oily phase (228) and an aqueous phase (230).

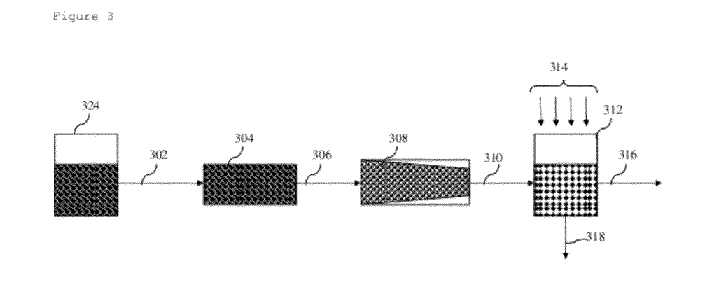

In FIG. 3 a filter residue (302) containing algae obtained from a storage vessel (324) is dried in drying unit (304). The dried filter residue (306) is forwarded to extruder (308), where the cell walls of the algae cells are disrupted to produce a lysate mixture (310). The lysate mixture (310) is forwarded to extraction unit (312), where algae oil is extracted from the lysate mixture by means of an extractive solvent (314). A mixture of the extractive solvent and the microbial oil (316) is obtained from a first outlet of the extraction unit (312), whilst algae residue comprising disrupted cell walls of algae cells (318) is obtained from a second outlet of the extraction unit (312).

The invention is further illustrated by the following non-limiting example.

### Example 1

About 760 kg of an aqueous mixture comprising diatomic algae and having a dry matter content of about 1 wt %, based on the total weight of the mixture, was coagulated and flocculated with the help of 10 ppm weight of a flocculant (Nalco 71303) to produce a coagulated and flocculated slurry of water and algae. The slurry was forwarded via a peristaltic pump to a dissolved air flotation (DAF) unit where air was blown into the slurry at a speed of 5 liters per minute at 4 bar (=20 NL/min.). A concentrated aqueous slurry was skimmed from the top of mixture in the DAF unit. The concentrated aqueous slurry had a dry matter content of about 4 wt %, based on the total weight of the slurry. About 25 kg of this concentrated aqueous slurry was subsequently fed to a prefiltering device comprising a polymer filter having a pore diameter of about 170 micron. Subsequently the slurry was fed onto a deformable filter situated in a so called dynamic belt filter press. The deformable filter comprised a polypropylene filter having a mesh size of about 100 micron, that is retaining particles having a diameter or equal to or more than about 100 micrometer. The deformable filter was mechanically deformed in an intermittent manner by means of peristaltic movements and was further forwarded via a conveyer belt. The conveyer belt was forwarded with a speed of about 10 cm/second. A pressure of about 6 bar was applied to press liquid out of the slurry through the deformable filter. From the dynamic filter press a cake was obtained having a dry matter content of about 31 wt %, based on the total weight of the mixture.

In table 1 the energy consumption per cubic meter of water removed is estimated for the above described process and a process where a mixture is concentrated by means of a centrifuge. Estimations were made on the basis of data available for the FEUX 420 centrifuge of Alfa-Laval and the Dewa H-PD belt filter press.

TABLE 1
Energy consumption

| | | | Dynamic filter |
|---|---|---|---|
| Parameter | | Centrifuge | press |
| Inlet range | wt % solids | 5 | 1-5 |
| Outlet range | wt % solids | 30 | 24-30+ |
| Energy/m3 water | kWh/m3 | 1-1.65 | 0.45 |
| removed | | | |

What is claimed is:

1. A process for separating a mixture containing a microbial substance and a liquid, wherein the microbial substance comprises at least one microorganism that store and/or secrete ethanol or the microbial substance comprises at least one whole oleaginous microorganism and/or is obtained after lysis of at least one oleaginous microorganism, comprising:
a) bringing the mixture in contact with a deformable filter;
b) filtering the mixture with such deformable filter to form a filtrate depleted in microbial substance and a filter residue enriched in microbial substance;
c) breaking the filter residue by deformation of the filter while the mixture is in contact with said filter;
d) separating the filtrate depleted in microbial substance and the filter residue enriched in microbial substance.

2. The process of claim 1 wherein the mixture consists essentially of the microbial substance and a liquid.

3. The process of claim 1 wherein the mixture is selected from the group consisting of fermentation broth, a lysate mixture, and a combination thereof.

4. The process of claim 1 wherein the mixture essentially consists of a liquid and one or more microorganisms that store and/or secrete ethanol.

5. The process of claim 4 wherein the microorganism comprise an ethanol-secreting yeast or an ethanol-secreting bacterium.

6. The process of claim 1 wherein the liquid contains water and/or ethanol.

7. The process of claim 1 wherein the mixture is subjected to a concentration step before step a).

8. The process of claim 7 wherein the mixture is concentrated by coagulation and/or flocculation before being contacted with the filter in step a).

9. The process of claim 1 wherein the filter is shaped as an essentially horizontally arranged belt or as an essentially horizontally arranged tube.

10. The process of claim 1 wherein the filter residue is broken by a mechanical deformation of the filter.

11. The process of claim 10 wherein the deformation of the filter includes peristaltic movement of the filter.

12. The process of claim 1 wherein the filter is arranged to move continuously over a conveyer belt.

13. A process for separating a mixture containing a microbial substance, water and/or ethanol, comprising:
i) bringing the mixture in contact with a deformable filter;
ii) filtering the mixture with such deformable filter to form a filtrate containing water and/or ethanol and a filter residue containing microbial substance;
iii) breaking the filter residue by deformation of the filter while the mixture is in contact with said filter;
iv) separating the filtrate and the filter residue.

14. The process of claim 13 wherein steps ii) and iii) are carried out continuously and/or simultaneously.
